(19) United States
(12) Patent Application Publication
(10) Pub. No.: US 2019/0352250 A1
(43) Pub. Date: Nov. 21, 2019

(54) PROCESS FOR THE PREPARATION OF OXALIC ACID ESTERS FROM CESIUM OXALATE

(71) Applicant: SABIC Global Technologies B.V., Bergen op Zoom (NL)

(72) Inventors: Ahmad AL-JABER, Thuwal (SA); Ilia KOROBKOV, Thuwal (SA); Khalid ALBAHILY, Thuwal (SA); Balamurugan VIDJAYACOUMAR, Thuwal (SA)

(21) Appl. No.: 16/475,476

(22) PCT Filed: Jan. 26, 2018
(86) PCT No.: PCT/IB2018/050500

§ 371 Date: Jul. 2, 2019

Related U.S. Application Data
(60) Provisional application No. 62/451,969, filed on Jan. 30, 2017.

(51) Int. Cl. C07C 67/08; C07C 51/41

(52) U.S. Cl. CPC C07C 67/08; C07C 51/41

(57) ABSTRACT

Processes for producing a disubstituted oxalate are disclosed. The process includes contacting a cesium salt with one or more alcohols and carbon dioxide (CO₂) under reaction conditions sufficient to produce a composition comprising a disubstituted oxalate.

### CROSS REFERENCE TO RELATED APPLICATIONS

This application claims the benefit of priority of U.S. Provisional Patent Application No. 62/451,969 filed Jan. 30, 2017, which is hereby incorporated by reference in its entirety.

### BACKGROUND OF THE INVENTION

### A. Field of the Invention

The invention generally concerns a process for preparing a disubstituted oxalate. In particular, the process includes contacting a cesium salt with one or more alcohols and carbon dioxide (CO₂) under reaction conditions sufficient to produce a disubstituted oxalate.

### B. Description of Related Art

Dimethyl oxalate (DMO) is the dimethyl ester of oxalic acid. DMO is used in various industrial processes, such as in pharmaceutical products, for the production of oxalic acid and ethylene glycol, or as a solvent or plasticizer. Commercially, DMO can be prepared by the high pressure oxidative coupling of carbon monoxide and an alkyl nitrite in the presence of a palladium catalyst. Most processes used to prepare DMO require carbon monoxide (CO) as a feedstock. CO is typically produced from the gasification of coal. Due to depleting global fossil fuel reserves, there is a foreseeable demand for new processes that require alternate feedstocks for DMO production.

### SUMMARY OF THE INVENTION

A discovery has been made that provides an alternate feedstock for the production of disubstituted oxalates (e.g., dimethyl oxalate). The discovery is premised on selectively converting cesium carbonate to cesium oxalate, and then reacting the cesium oxalate with methanol and CO₂ to produce the disubstituted oxalate. The production of disubstituted oxalates can be performed in a step-wise manner or in a single-pot fashion where cesium oxalate is generated in situ. The process of the current invention provides an elegant alternative to conventional methods of making dimethyl oxalate from CO and alkyl nitrites using expensive noble metal catalysts.

In one embodiment of the current invention, a process for producing a disubstituted oxalate is described. The process includes contacting a cesium salt (e.g., cesium oxalate) with one or more alcohols and carbon dioxide (CO₂) under reaction conditions sufficient to produce a composition containing a disubstituted oxalate having the general structure of:

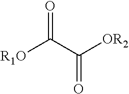

where R₁ and R₂ are each independently an alkyl group, a substituted alkyl group, an aromatic group, a substituted aromatic group, or a combination thereof. In one aspect, the reaction conditions can include a temperature of 125° C. to 200° C., 130° C. to 180° C., or preferably about 150° C. and/or a pressure of 2 MPa to 5 MPa, 3 MPa to 4 MPa, or preferably about 3.5 MPa. The cesium salt used in the process can be cesium oxalate. The cesium oxalate can be obtained by contacting a mixture of CO₂ and carbon monoxide (CO) under reaction conditions sufficient to form a composition containing the cesium oxalate. In some instances, the cesium oxalate can be obtained by contacting a mixture of CO₂ and hydrogen (H₂), or a mixture of O₂ and CO with cesium carbonate (Cs₂CO₃), under reaction conditions sufficient to form a composition containing the cesium oxalate. Particularly, the reaction conditions for obtaining the cesium oxalate can include a temperature of 200° C. to 400° C., 250° C. to 350° C., preferably 290° C. to 335° C., or most preferably 300° C. to 325° C. In some instances, the reaction conditions for obtaining the cesium oxalate can include providing carbon dioxide at a pressure of 2.0 MPa to 3.0 MPa, preferably about 2.5 MPa, and providing carbon monoxide at a pressure of 1.0 MPa to 3 MPa, preferably about 2.0 MPa. In other instances, the reaction conditions for obtaining the cesium oxalate can include providing carbon dioxide at a pressure of 2.0 MPa to 4.0 MPa, preferably about 3.5 MPa, and providing H₂ at a pressure of 0.05 MPa to 0.5 MPa, preferably about 0.1 MPa. In yet another instance, the reaction conditions for obtaining the cesium oxalate can include providing carbon monoxide at a pressure of 2.0 MPa to 4.0 MPa, preferably about 3.5 MPa, and providing O₂ at a pressure of 0.05 MPa to 4 MPa, 0.1 to 1.5 MPa, or about 0.1 MPa. The process can further include contacting the cesium carbonate with the carbon dioxide at a reaction temperature of 200° C. to 400° C., 250° C. to 350° C., preferably 290° C. to 335° C., or most preferably 300° C. to 325° C., for at least 1 hour to form a cesium carbonate/carbon dioxide reaction mixture and then contacting the cesium carbonate/carbon dioxide reaction mixture with hydrogen. Such a controlled addition of the carbon dioxide and hydrogen can inhibit the formation of sodium formate. In a particular instance, the process can further include isolating the cesium oxalate salt from the product stream prior to converting it to the disubstituted oxalate. Alternatively, the process can be a one-pot synthesis such that it is performed in a single reactor such that cesium oxalate is generated in situ and then contacted with the one or more alcohols and additional CO₂ to produce the disubstituted oxalate. In particular instances, R₁ and R₂ can include 1 to 20 carbon atoms, 1 to 10 carbon atoms, 1 to 5 carbon atoms, preferably 1 carbon atom. More specifically, R₁ and R₂ can be a methyl group, an ethyl group, a propyl group, an isopropyl group, a n-butyl group, a sec-butyl group, a tert-butyl group, a pentyl group, a neopentyl, or a hexyl group, or combinations thereof. In some aspects of the current invention, R₁ and R₂ are each methyl groups. In other aspects, the product stream can also include cesium bicarbonate. In certain aspects, the process can further include isolating the disubstituted oxalate from the product stream. In still other aspects, the process can include reacting the disubstituted oxalate under conditions sufficient to form oxalic acid or reacting the disubstituted oxalate under conditions sufficient to form ethylene glycol.

In another embodiment of the present invention, there is disclosed a composition for producing a disubstituted oxalate. The composition can include a cesium salt (e.g., cesium oxalate, and optionally, cesium carbonate or cesium bicarbonate), an alcohol, carbon dioxide, and, optionally, carbon monoxide. In one aspect, the alcohol can be methanol and the disubstituted oxalate can be dimethyl oxalate. In another aspect, a disubstituted oxalate is produced by any of the processes described in the current invention. In particularly preferred embodiments, the produced disubstituted oxalate is dimethyl oxalate (DMO).

The following includes definitions of various terms and phrases used throughout this specification.

The term “alkyl group” can be a straight or branched chain alkyl having 1 to 20 carbon atoms. Examples include methyl, ethyl, propyl, isopropyl, butyl, isobutyl, secondary butyl, tertiary butyl, pentyl, isopentyl, neopentyl, hexyl, benzyl, heptyl, octyl, 2-ethylhexyl, 1,1,3,3-tetramethylbutyl, nonyl, decyl, dodecyl, tetradecyl, hexadecyl, octadecyl, and/or eicosyl.

The term “substituted alkyl group” can include any of the aforementioned alkyl groups that are additionally substituted with one or more heteroatom, such as a halogen (F, Cl, Br, I), boron, oxygen, nitrogen, sulfur, silicon, etc. Without limitation, a substituted alkyl group can include alkoxy or alkylamine groups where the alkyl group attached to the heteroatom can also be a substituted alkyl group.

The term “aromatic group” can be any aromatic hydrocarbon group having 5 to 20 carbon atoms of the monocyclic, polycyclic or condensed polycyclic type. Examples include phenyl, biphenyl, naphthyl, and the like. Without limitation, an aromatic group also includes heteroaromatic groups, for example, pyridyl, indolyl, indazolyl, quinolinyl, isoquinolinyl, and the like.

The term “substituted aromatic group” can include any of the aforementioned aromatic groups that are additionally substituted with one or more atom, such as a halogen (F, Cl, Br, I), carbon, boron, oxygen, nitrogen, sulfur, silicon, etc. Without limitation, a substituted aromatic group can be substituted with alkyl or substituted alkyl groups including alkoxy or alkylamine groups.

The terms “about” or “approximately” are defined as being close to as understood by one of ordinary skill in the art. In one non-limiting embodiment, the terms are defined to be within 10%, preferably, within 5%, more preferably, within 1%, and most preferably, within 0.5%.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. With respect to the transitional phase “consisting essentially of,” in one non-limiting aspect, a basic and novel characteristic of the process of the present invention is the ability to produce a disubstituted oxalate from CO₂, CO, and an alcohol.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 is the CO to CO₂ transformation energies.

FIG. 2 is the Cs₂CO₃ to Cs₂(C₂O₄) transformation energies.

FIG. 3 is the Cs₂C₂O₄ to DMO transformation energies.

FIG. 4 is the Cs₂CO₃ regeneration from CsOH transformation energies.

FIG. 5 is a schematic of a one reactor system to produce disubstituted oxalates of the present invention.

FIG. 6 is a schematic of a two reactor system to produce disubstituted oxalates of the present invention.

While the invention is susceptible to various modifications and alternative forms, specific embodiments thereof are shown by way of example in the drawings. The drawings may not be to scale.

### DETAILED DESCRIPTION OF THE INVENTION

A discovery has been made that provides an elegant solution to the problem of diminishing carbon monoxide (CO) feedstocks that are used for producing disubstituted oxalates such as dimethyl oxalate. The discovery is premised on contacting a cesium salt (e.g., cesium oxalate) with one or more alcohols and carbon dioxide (CO₂) under reaction conditions sufficient to produce a disubstituted oxalate (e.g., dimethyl oxalate) containing composition as shown in overall general reaction equation (1).

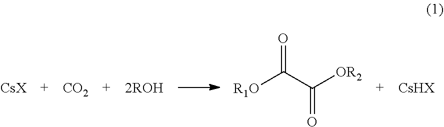

where X is a counter anion to the cesium metal cation and ROH can be the same or different alcohols and R₁ and R₂ are defined as above. In a preferred embodiment, ROH is methanol and the disubstituted oxalate is dimethyl oxalate. In some instances, the cesium salt (CsX) is cesium oxalate. These and other non-limiting aspects of the present invention are discussed in further detail in the following sections with reference to the Figures.

### A. Cesium Oxalate

In particular embodiments, the process of the present invention provides temperature efficient and alternative processes for the formation of cesium oxalate. Cesium carbonate can be selectively converted to cesium oxalate though the reaction with CO₂ and CO. The cesium carbonate can be supported (e.g., alumina or silica support) or be used in an unsupported form (i.e., bulk catalyst). Alternative processes to produce cesium carbonate include the reaction of CO₂ and H₂, or the reaction of CO and O₂, under sufficient temperature and pressures to produce cesium oxalate. In a non-limiting aspect, the formed cesium oxalate can be further reacted in situ or separately to form further synthesis products (e.g., disubstituted oxalate).

Conventionally, cesium oxalate is generated by the reaction of cesium carbonate with carbon monoxide and carbon dioxide as shown in reaction equation (2).

Cs₂CO₃+CO+CO₂→Cs₂(C₂O₄) (2).

In one alternative process, the cesium oxalate can be generated by the reaction of cesium carbonate with carbon dioxide and H₂ as shown in reaction equation (3) as described in more detail below and in the Examples section.

Cs₂CO₃+O₂+CO→Cs₂(C₂O₄) (3).

In some embodiments, the carbon dioxide and H₂ are added in a sequential manner as shown in reaction equation (4). The sequential addition of carbon dioxide then hydrogen can inhibit or substantially inhibit the formation of cesium formate (HCO₂Cs). Limiting the formation of cesium formate, limits the formation of alkyl formate in subsequent reactions with alcohols. In some instances, cesium formate is not formed in the production of cesium oxalate.

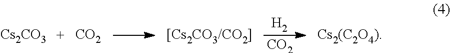

In yet another alternative process, the cesium oxalate can be generated by the reaction of cesium carbonate with carbon monoxide and O₂ as shown in reaction equation (5) as described in more detail below.

Cs₂CO₃+O₂+CO→Cs₂(C₂O₄) (5).

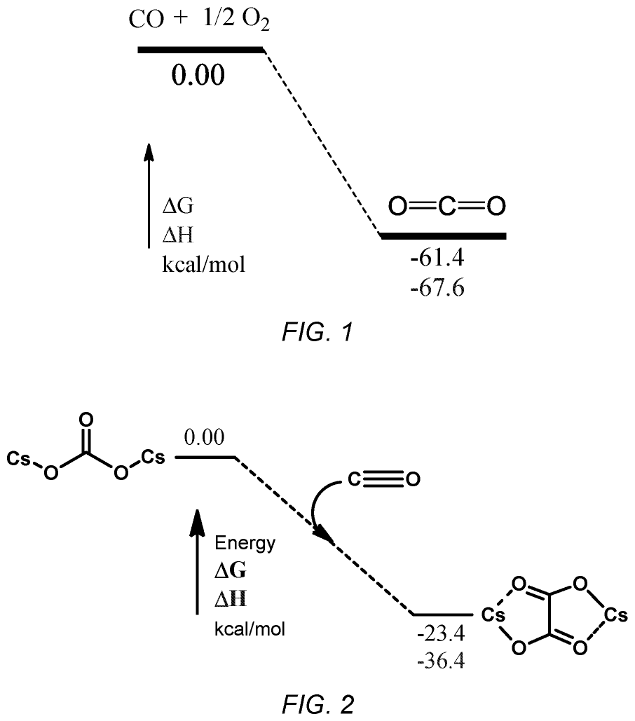

With respect to reaction equation (5), and without wishing to be bound by theory, it is believed that the use of molecular oxygen will require lower heat requirements when compared to other processes as the reaction between CO and O₂ is exothermic (free energy change of −61.4 kcal/mol as determined through density functional theory (DFT)). FIG. 1 depicts the carbon monoxide to carbon dioxide transformation energetics. The CO₂ can bind with cesium carbonate to form a CO₂—Cs₂CO₃ adduct, which has an enthalpy of fusion at a molecular level. This enthalpy of fusion can be compensated by the CO+0.5 O₂ to CO₂ energy of 122.8 kcal/mol. The remaining carbon monoxide can then transform the CO₂—Cs₂CO₃ adduct into cesium oxalate. FIG. 2 shows the overall Cs₂CO₃ to Cs₂C₂O₄ transformation energies. Thus, the overall reaction is exothermic with a calculated free energy (DFT) change of −23.4 kcal/mol making the reaction favorable for low heating requirements.

### B. Disubstituted Oxalate

The produced cesium oxalate product from Section A can then be reacted with the desired alcohol in the presence of carbon dioxide to produce the desired disubstituted oxalate. In some instances, the produced cesium oxalate product is first purified before being converted to a disubstituted oxalate. Such purification may help with reducing or avoiding the formation of undesired by-products during disubstituted oxalate production. Reaction equations (6) through (8) show the overall reaction starting with cesium salt under a conventional CO/CO₂ atmosphere (reaction equation (6)), and the alternative processes using H₂/CO₂ (reaction equation (7)), or CO/O₂ (reaction equation (8)). Reaction conditions are described in more detail below and in the Examples Section.

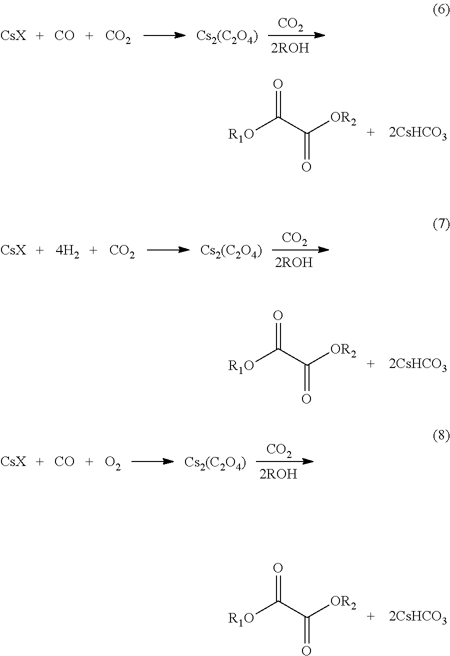

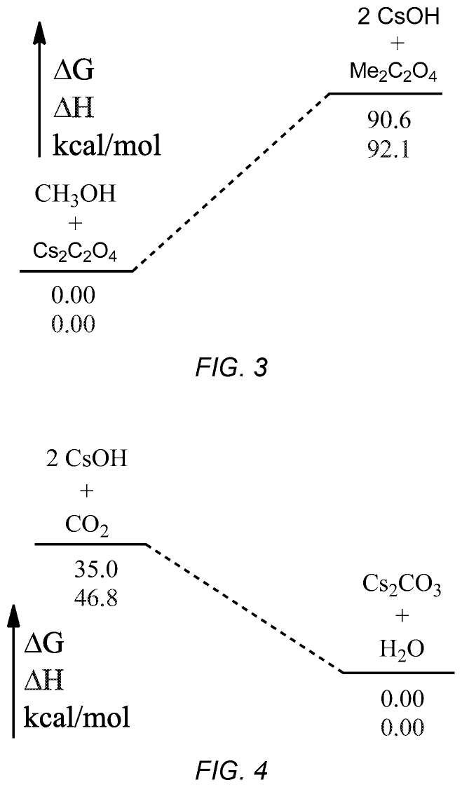

Without wishing to be bound by theory, it is believed that the conversion of cesium oxalate to disubstituted oxalates (e.g., dimethyl oxalate (DMO)) is endothermic with an overall calculated free energy (DFT) change of about 91 kcal/mol. For example, FIG. 3 shows Cs₂C₂O₄ to DMO transformation energies. Thus, the exothermic formation of cesium oxalate from cesium carbon monoxide and oxygen can provide energy for this step, thereby requiring less overall energy (e.g., heat input).

### C. Sustainability

Under certain conditions, cesium hydroxide (CsOH), unreacted cesium oxalate, and/or the cesium bicarbonate can be formed. These products can be separated or further processed. By way of example cesium hydroxide can be isolated and converted into cesium carbonate, thereby regenerating the cesium catalyst. At the molecular level this reaction is exothermic with a calculated free energy (DFT) change of about 35 kcal/mol. FIG. 4 shows Cs₂CO₃ regeneration from CsOH transformation energies. The overall sustainable process is showed in the schematic below. As discussed above and throughout this specification, the combination of “reactant 1” and “reactant 2” in the schematic can be a combination of CO₂+CO, CO₂+H₂, or CO+O₂.

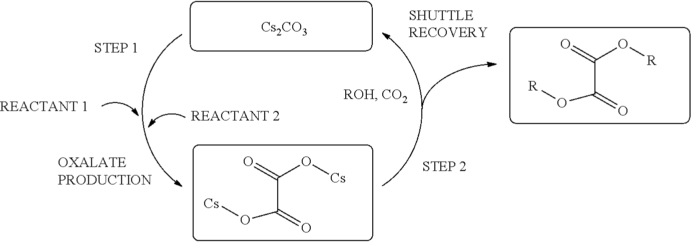

### D. System and Processes to Prepare Cesium Oxalate and Disubstituted Oxalate

1. Single Reactor Preparation of Cesium Oxalate and Disubstituted Oxalate

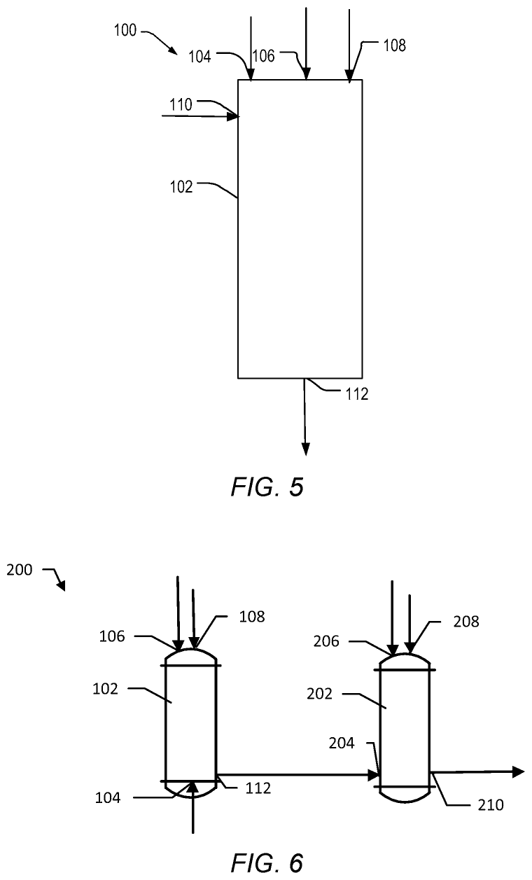

Any of the processes of the present invention can be performed in a single reactor. Referring to FIG. 5, a method and system to prepare disubstituted oxalates is described. In system 100, cesium salt precursor (e.g., cesium carbonate (Cs₂CO₃)) can be provided to a reactor unit 102 via solids inlet 104. CO, CO₂, O₂, H₂ or any combination thereof can be provided to reactor 102 via gas inlets 106 and 108. By way of example, CO₂ can be provided to reactor 102 via gas inlet 108 and CO or H₂, can be provided to the reactor via gas inlet 106. CO can be provided to reactor 102 via gas inlet 106 and O₂ can be provided to the reactor via gas inlet 108. In embodiments when carbon monoxide is used, the CO can be provided to reactor 102 at a pressure ranging from 1 MPa to 3 MPa and all ranges and pressures there between (e.g., 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, or 2.9 MPa). Preferably, the CO pressure is about 2 MPa. In other embodiments when H₂ is used, the H₂ can be provided to reactor 102 at a pressure ranging from 0.05 MPa to 0.5 MPa, 0.05 to 0.4 MPa, 0.05 to 0.3 MPa, 0.05 to 0.2 MPa, or 0.05 to 0.1 and all ranges and pressures there between (e.g., 0.05 MPa, 0.06 MPa, 0.07 MPa, 0.08 MPa, 0.09 MPa, 0.1 MPa, 0.11 MPa, 0.12 MPa, 0.13 MPa, 0.14 MPa, 0.15 MPa, 0.16 MPa, 0.17 MPa, 0.18 MPa, 0.19 MPa, 0.20 MPa, 0.21 MPa, 0.22 MPa, 0.23 MPa, 0.24 MPa, 0.25 MPa, 0.26 MPa, 0.27 MPa, 0.28 MPa, 0.29 MPa, 0.30 MPa, 0.31 MPa, 0.32 MPa, 0.33 MPa, 0.34 MPa, 0.35 MPa, 0.36 MPa, 0.37 MPa, 0.38 MPa, 0.39 MPa, 0.40 MPa, 0.41 MPa, 0.42 MPa, 0.43 MPa, 0.44 MPa, 0.45 MPa, 0.46 MPa, 0.47 MPa, 0.48 MPa, 0.49 MPa, or 0.50 MPa). Preferably, the H₂ pressure is about 0.1 MPa. In other embodiments when O₂ is used, the O₂ can be provided to reactor 102 at a pressure ranging from 0.05 MPa to 4 MPa, 0.1 to 1.5 MPa, or about 0.1 MPa. CO₂ can be provided to reactor 102 at a pressure ranging from 1 MPa to 4 MPa and all ranges and pressures there between (e.g., 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, 2.9 MPa, 3.0 MPa, 3.1 MPa, 3.2 MPa, 3.3 MPa, 3.4 MPa, 3.5 MPa, 3.6 MPa, 3.7 MPa, 3.8 MPa, 3.9 MPa, or 4 MPa). Preferably, the CO₂ pressure is about 2.5 MPa to 3.5 MPa. The upper limit on pressure can be determined by the type and size of reactor used. Although not shown, in some embodiments, CO₂ CO, O₂, or H₂, and can be provided to reactor unit 102 via the same inlet. In certain embodiments, mixtures of CO₂, CO, O₂, and H₂ are used. By way of example, CO₂ can be used with CO, CO₂ can be used with H₂, CO, or CO and H₂, and CO can be used with O₂. Reactor 102 can be pressurized either through the addition of the gases and/or with an inert gas. The average pressure of reactor unit 102 ranges from 2.0 to 4 MPa (e.g., 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 MPa) after charging the CO₂. Reactor 102 can be heated to a temperature sufficient to promote the reaction of cesium carbonate with the carbon dioxide and carbon monoxide or H₂ to produce a product composition that includes cesium oxalate. The temperature range of the reactor 102 can be 200° C. to 400° C., 250° C. to 350° C., and all ranges and temperatures there between (e.g., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., 320° C., 325° C., 330° C., 335° C., 340° C., 345° C., 350° C., 355° C., 360° C., 365° C., 370° C., 375° C., 380° C., 385° C., 390° C., or 395° C.). Preferably, the reaction temperature is 290° C. to 335° C., or most preferably 300° C. to 325° C. The reactants can be heated for a time sufficient to react all or a substantially all of the cesium carbonate. By way of example, the reaction time range can be at least 1 hour, 1 to 5 hours, 1 hours to 4 hours, 1 hour to 3 hours, and all ranges and times there between (e.g., 1.25 hours, 1.5 hours, 1.75 hours, 2 hour, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours). When CO is used, the reaction time can be about 2 hours. When H₂ is used, the cesium carbonate can be reacted with the carbon dioxide for 1 to 3 hours, (e.g., 1, 1.5, 2, 2.5, 3 hours), and then with H₂ for an additional 1 to 3 hours, (e.g., 1, 1.5, 2, 2.5, 3 hours).

Reactor 102 can be cooled and/or depressurized to a temperature and pressure sufficient to add the desired alcohol. By way of example, reactor 102 can be cooled to a temperature range of 100° C. to 160° C., or 130° C. to 150° C., or about 150° C. at a pressure of 0.101 MPa to 1 MPa. The desired alcohol (e.g., methanol) can be added to reactor 102 via liquid inlet 110 to form a composition that includes a cesium salt (e.g., cesium oxalate, and optionally, cesium carbonate and/or cesium bicarbonate), an alcohol, carbon dioxide, and, optionally, carbon monoxide. The reactor can be pressurized with carbon dioxide and/or an inert gas to a pressure ranging from 2 MPa to 5 MPa, 3 MPa to 4 MPa, and all ranges and pressures there between (e.g., 2.1 MPa, 2.2 MPa, 2.3 MPa, 2.4 MPa, 2.5 MPa, 2.6 MPa, 2.7 MPa, 2.8 MPa, 2.9 MPa, 3 MPa, 3.1 MPa, 3.2 MPa, 3.3 MPa, 3.4 MPa, 3.5 MPa, 3.6 MPa, 3.7 MPa, 3.8 MPa, 3.9 MPa, 4.0 MPa, 4.1 MPa, 4.2 MPa, 4.3 MPa, 4.4 MPa, 4.5 MPa, 4.6 MPa, 4.7 MPa, 4.8 MPa, or 4.9 MPa). In some embodiments, carbon dioxide is present in sufficient amounts that additional CO₂ is not necessary.

After the addition of the alcohol, and, optionally, CO₂, the reactor can be heated to a reaction temperature sufficient to promote the cesium oxalate salt to react with the alcohol under the carbon dioxide atmosphere to produce a disubstituted oxalate containing composition. In other embodiments, sufficient carbon dioxide remains in reactor 102. The reaction temperature can be 125° C. to 225° C., 130° C. to 180° C., and all ranges and temperatures there between (e.g., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., or 220° C.). In some instances, the reaction temperature is about 150° C. Reactor 102 can be heated for a time sufficient to react all or substantially all of the cesium salt (e.g., cesium oxalate). By way of example, the reaction time range can be at least 1 hour, 1 hours to 18 hours, 10 hour to 14 hours, 1 to 6 hours or 1 to 2 hours, and all ranges and times there between (e.g., 2 hours, 5 hours, 8 hours, 10 hours, 15 hours, or 17 hours). Preferably, the reaction time is 1 to 18 hours, or 15 hours. The upper limit on temperature, pressure, and/or time can be determined by the reactor used. The disubstituted oxalate reaction conditions can be further varied based on the type of the reactor used.

Reactor 102 can be cooled and depressurized to a temperature and pressure sufficient (e.g., below 50° C. at 0.101 MPa) to allow removal of the product composition containing disubstituted oxalate via product outlet 112. The product composition can be collected for further use. In some instances, the product composition can include cesium bicarbonate (CsHCO₃).

### 2. Two Reactors

In some embodiments, reactor 102 can be depressurized and cooled to a temperature sufficient to allow the cesium oxalate containing product composition to be removed from the reactor via product outlet 112. The product composition can be further treated (e.g., washed) to remove any unreacted products. In one embodiment, the product composition is used without purification. The cesium oxalate can then be transferred to a second reactor unit to produce disubstituted oxalates. Referring to FIG. 2, a schematic of system 200 having two reactor units is depicted. The cesium salt precursor (e.g., cesium carbonate) can be provided to reactor 102 via inlet 104 and contacted with carbon dioxide in combination with carbon monoxide and/or H₂ or the combination of carbon monoxide and oxygen as described above (See, FIG. 1) to generate the cesium oxalate.

The cesium oxalate can exit reactor 102 via product outlet 112 and enter reactor 202 via cesium oxalate inlet 204. The desired alcohol can be provided to reactor 202 via alcohol inlet 206. Carbon dioxide can be provided to reactor 208 via carbon dioxide inlet 208. Reactor 202 can be pressurized to a pressure of 2.0 to 5 MPa (e.g., 2.0, 2.1, 2.2, 2.3, 2.4, 2.5, 3.0, 3.5, 4.0, 4.5, 5.0 MPa) either by the addition of the carbon dioxide or using an inert gas. Once reactor 202 has been pressurized, heat can be applied to the reactor using known methods (e.g., electrical heaters, heat transfer medium, or the like) to a temperature sufficient to promote the reaction of cesium oxalate and the alcohol. The reaction temperature can be 125° C. to 225° C., 130° C. to 180° C., and all ranges and temperatures there between (e.g., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., or 220° C.). In some instances, the reaction temperature is about 150° C. Reactor 202 can be heated for a time sufficient to react all or substantially all of the cesium salt (e.g., cesium oxalate). By way of example, the reaction time range can be at least 1 hour, or 1 to 18 hours, 1 hour to 16 hours, 10 hour to 14 hours, and all ranges and times there between as previously described. Preferably, the reaction time is about 1 hour to 18 hours, or about 15 hours. The upper limit on temperature, pressure, and/or time can be determined by the reactor used. The disubstituted oxalate reaction conditions may be further varied based on the type of the reactor used.

Reactor 202 can be cooled and depressurized to a temperature and pressure sufficient (e.g., below 50° C. at 0.101 MPa) to allow removal of the product composition containing disubstituted oxalate via product outlet 210. The product composition can be collected for further use or sale.

Reactors 102 and 202 and associated equipment (e.g., piping) can be made of materials that are corrosion and/or oxidation resistant. By way of example, the reactor can be lined with, or made from, Inconel. The design and size of the reactor is sufficient to withstand the temperatures and pressures of the reaction. The systems can include various automated and/or manual controllers, valves, heat exchangers, gauges, etc., for the operation of the reactor, inlets, and outlets. The reactor can have insulation and/or heat exchangers to heat or cool the reactor as desired. Non-limiting examples of a heating/cooling source can be a temperature controlled furnace or an external, electrical heating block, heating coils, or a heat exchanger. The reaction can be performed under inert conditions such that the concentration of oxygen (O₂) gas in the reaction is low or virtually absent in the reaction such that O₂ has a negligible effect on reaction performance (i.e., conversion, yield, efficiency, etc.).

### E. Reactants and Products

CO₂ gas, CO gas, O₂ gas, and H₂ gas can be obtained from various sources. In one non-limiting instance, the CO₂ can be obtained from a waste or recycle gas stream (e.g., from a plant on the same site such as from ammonia synthesis, or a reverse water gas shift reaction) and/or after recovering the carbon dioxide from a gas stream. A benefit of recycling carbon dioxide as a starting material in the process of the invention is that it can reduce the amount of carbon dioxide emitted to the atmosphere (e.g., from a chemical production site). The CO can be obtained from various sources, including streams coming from other chemical processes, like partial oxidation of carbon-containing compounds, iron smelting, photochemical process, syngas production, reforming reactions, and/or various forms of combustion. O₂ can come from various sources, including streams from water-splitting reactions and/or cryogenic separation systems. The hydrogen may be from various sources, including streams coming from other chemical processes, like water splitting (e.g., photocatalysis, electrolysis, or the like), syngas production, ethane cracking, methanol synthesis, and/or conversion of methane to aromatics. In some embodiments, the gases are obtained from commercial gas suppliers. When a mixture of gases is used such as a mixture of CO₂ and CO or H₂, or CO and O₂, the gas can be premixed or mixed when added separately to the reactor. When the reactor contains a mixture of CO₂ and CO, the pressure ratio of CO₂:CO in the reactor can be greater than 0.1. In some embodiments, the CO₂:CO pressure ratio can be from 0.2:1 to 5:1, from 0.5:1 to 2:1, or 1:1 to 1.5:1. Preferably, the CO₂:CO pressure ratio is about 1.25. The partial pressure at room temperature ratio of CO₂:CO in the reactor can range from 40:10 or from 45:15. When the reactor contains a mixture of CO₂ and H₂, the pressure ratio of CO₂:H₂ in the reactor can be greater than 0.1. In some embodiments, the CO₂:H₂ ratio can be from 5:1 to 80:1, from 10:1 to 60:1, 20:1 to 50:1, or 30:1 to 40:1, or 35:1. Preferably, the CO₂:H₂ pressure ratio is about 35:1. The partial pressure at room temperature of CO₂:H₂ in the reactor can range from 4.5 MPa to 1 MPa, or from 1 MPa to 0.1 MPa. When the reactor contains a mixture of CO and O₂, the pressure ratio of CO:O₂ in the reactor can be greater than 0.1. In some embodiments, the CO:O₂ pressure ratio can be from 5:1 to 80:1, from 10:1 to 60:1, 20:1 to 50:1, or 30:1 to 40:1, or 35:1. In another example, cesium carbonate is contacted with CO and O₂ to form cesium oxalate. The pressure ratio of CO and O₂ to cesium carbonate can be 1:0.5 to 3:1 and all ranges and values there between (e.g., 1:0.5, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.6, 1:2.7, 1:2.8, or 1:2.9) Preferably the ratio is 2:1. In some examples, the remainder of the reactant gas can include another gas or gases provided the gas or gases are inert, such as argon (Ar) and/or nitrogen (N₂), further provided that they do not negatively affect the reaction. Preferably, the reactant mixture is highly pure and substantially devoid of water. In some embodiments, the gases can be dried prior to use (e.g., pass through a drying media) or contain a minimal amount of water or no water at all. Water can be removed from the reactant gases with any suitable method known in the art (e.g., condensation, liquid/gas separation, etc.).

Alcohols may be purchased in various grades from commercial sources. Non-limiting examples of the alcohol that can be used in the process of the current invention to form a disubstituted oxalate can include methanol, ethanol, n-propanol, isopropanol, n-butanol, isobutanol, sec-butanol, tert-butanol, 1-pentanol, 2-pentanol, 3-pentanol, 3-methyl-1-butanol, 2-methyl-1-butanol, 2,2-dimethyl-1-propanol, 3-methyl-2-butanol, 2-methyl-2-butanol, hexanol, 2-hexanol, 3-hexanol, 1-heptanol, 2-heptanol, 3-heptanol, 4-heptanol, 1-octanol, 2-octanol, 3-octanol, 4-octanol, cyclohexanol, cvclopentanol, phenol, benzyl alcohol, ethylene glycol, propylene glycol, or butylene glycol or any combination thereof. In certain embodiments, the alcohol includes a mixture of stereoisomers, such as enantiomers and diastereomers. Preferably, the alcohol is methanol, ethanol, n-propanol, isopropanol, n-butanol, isobutanol, .sec.-butanol, tert-butanol, 1-pentanol, 2,2-dimethyl-1-propanol (neopentanol), hexanol, or combinations thereof.

Cesium carbonate (Cs₂CO₃) may be purchased in various grades from commercial sources. Preferably, the alcohol and Cs₂CO₃ are highly pure and substantially devoid of water. A non-limited commercial source of the alcohols and Cs₂CO₃ for use in the present invention includes Sigma-Aldrich®, (USA). In some embodiments, Cs₂CO₃ is mixed with an inert material. Non-limiting examples of inert materials include alumina (acidic, basic or neutral), silica, zirconia, ceria, zeolites, lanthanum oxides, or mixtures thereof. In preferred embodiments, the Cs₂CO₃ is mixed with alumina or silica using solid-solid mixing. Providing the Cs₂CO₃ as a Cs₂CO₃/inert material mixture can inhibit the cesium oxalate from forming a melt that requires further processing (e.g., grinding, powdering, etc.) prior to reaction with alcohol to form the disubstituted oxalate of the present invention.

The process of the present invention can produce a product stream that includes a composition containing a disubstituted oxalate and optionally cesium bicarbonate (CsHCO₃) that can be suitable as an intermediate or as feed material in a subsequent synthesis reactions to form a chemical product or a plurality of chemical products (e.g., such as in pharmaceutical products, for the production of oxalic acid and ethylene glycol, or as a solvent or plasticizer). In some instances, the composition containing a disubstituted oxalate can be directly reacted under conditions sufficient to form oxalic acid or ethylene glycol. The product composition includes at least 50 wt. %, at least 60 wt. %, at least 70 wt. %, at least 80 wt. %, at least 90 wt. % or 100 wt. % disubstituted oxalate, with the balance being cesium bicarbonate. The product composition can be purified using known organic purification methods (e.g., extraction, crystallization, distillation washing, etc.) depending on the phase of the production composition (e.g., solid or liquid). In a preferred embodiment, the disubstituted oxalate can be recrystallized from hot alcohol (e.g., methanol) solution. DMO can be purified by distillation (boiling point of 166° C.) or crystallization (melting point 54° C.).

The disubstituted oxalate produced by the process of the present invention can have the general structure of:

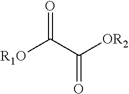

where R₁ and R₂ can be each independently alkyl group, a substituted alkyl group, an aromatic group, a substituted aromatic group, or a combination thereof. R₁ and R₂ can include 1 to 20 carbon atoms, 1 to 10 carbon atoms, 1 to 5 carbon atoms, preferably 1 carbon atom. Non-limiting examples of R₁ and R₂ include methyl, ethyl, n-propyl, isopropyl, n-butyl, isobutyl, sec-butyl, tert-butyl, 1-pentyl, 2-pentyl, 3-pentyl, 3-methyl-1-butyl, 2-methyl-1-butyl, 2,2-dimethyl-1-propyl, 3-methyl-2-butyl, 2-methyl-2-butyl, 1-hexyl, 2-hexyl, 3-hexyl, 1-heptyl, 2-heptyl, 3-heptyl, 4-heptyl, 1-octyl, 2-octyl, 3-octyl, 4-octyl, cyclohexyl, cyclopentyl, phenyl, or benzyl. Preferably, R₁ and R₂ are a methyl group, an ethyl group, a propyl group, an isopropyl group, a n-butyl group, a sec-butyl group, a tert-butyl group, a pentyl group, a neopentyl, a hexyl group, or combinations thereof. In certain embodiments, R₁ and R₂ can include a mixture of stereoisomers, such as enantiomers and diastereomers. In a specific embodiment, the disubstituted oxalate is a dialkyl oxalate, such as dimethyl oxalate (DMO) where R₁ and R₂ are each methyl groups.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

Cesium carbonate (Cs₂CO₃) was obtained from Sigma-Aldrich® (U.S.A) in powder form and 99.9% purity. Methanol was obtained from Fisher Scientific (HPLC grade, U.S.A.) in 99.99% purity. ¹³C NMR was performed on a 400 MHz Bruker instrument (Bruker, U.S.A). The Parr reactor used was obtained from Parr Instrument Company, USA.

### Example 1

### Two-Step Process for the Preparation of Dimethyl Oxalate with CO and CO₂

Cs₂CO₃ (500 mg, 0.15 mmol) was added to a 100 mL Parr reactor in a glove box. CO₂ (25 bar) and CO (20 bar) gases were then charged and the mixture was stirred for 1-2 hour at 300° C. and cooled to room temperature by circulating air around the reactor. The reactor was depressurized. The product obtained was a solid and a portion was removed from the reactor as a soft (molten) solid. ¹³C NMR analysis was performed on the salt, and confirmed that the salt was primarily cesium oxalate. Methanol (5 mL) was then added to the reactor, and the reactor was pressurized with CO₂ (35 bar). The mixture was heated to 150° C., stirred overnight, and then depressurized. The remaining solvent (methanol) was removed by evaporation under vacuum. The product composition was analyzed and identified as being a mixture of dimethyl oxalate, cesium formate, and cesium bicarbonate. The overall yield of DMO was 94% and the yield of cesium formate as a byproduct was 4% to 5%. ¹³C NMR (CD₃OD, in ppm): 53 (—OMe), 158 (—CO—), 161 (CsHCO₃), and 171 (CsHCOO).

### Example 2

### One-Step Process for the Preparation of Dimethyl Oxalate with CO

Cs₂CO₃ (500 mg, 0.15 mmol) was added to a 100 mL Parr reactor in a glove box. CO₂ (35 bar, 3.5 MPa) and H₂ (1 bar, 0.1 MPa) gases were then charged and the mixture was stirred for 1-2 hour at 325° C. and cooled to room temperature by applying cool air to the reactor. The reactor cooled to 25° C. and depressurized. The reaction mixture contained cesium oxalate, cesium formate, and cesium bicarbonate. Methanol (5 mL) was then added to the reactor, and the reactor was pressurized with CO₂ (35 bar, 3.5 MPa). The mixture was heated to 150° C., stirred overnight, and then depressurized. The remaining solvent (methanol) was removed by evaporation under vacuum. The product composition was analyzed and identified as being a mixture of dimethyl oxalate, cesium formate, and cesium bicarbonate. The overall yield of DMO was 89% and the yield of cesium formate as a byproduct was about 4% to 5%. ¹³C NMR (CD₃OD, in ppm): 53 (—OMe), 158 (—CO—), 161 (CsHCO₃), and 171 (CsHCOO).

### Example 3

### Two-Step Process for the Preparation of Dimethyl Oxalate with 112

Cs₂CO₃ (500 mg, 0.15 mmol) was added to a 100 mL Parr reactor in a glove box. CO₂ (35 bar, 3.5 MPa) and H₂ (1 bar, 0.1 MPa) gases were then charged and the mixture was stirred for 1-2 hour at 325° C. and cooled to room temperature by applying cool air to the reactor. The reactor cooled to 25° C. and was depressurized. The reaction mixture contained cesium oxalate, cesium formate, and cesium bicarbonate. Methanol (5 mL) was then added to the reactor, and the reactor was pressurized with CO₂ (35 bar, 3.5 MPa). The mixture was heated to 150° C., stirred overnight, and then depressurized. The remaining solvent (methanol) was removed by evaporation under vacuum. The product composition was analyzed and identified as being a mixture of dimethyl oxalate, cesium formate, and cesium bicarbonate. The overall yield of DMO was 54% and the yield of cesium formate as a byproduct was about 4% to 5%. ¹³C NMR (CD₃OD, in ppm): 53 (—OMe), 158 (—CO—), 161 (CsHCO₃), and 171 (CsHCOO).

### Example 4

### Two-Step Process for the Preparation of Dimethyl Oxalate with Sequential Addition of 112

Cs₂CO₃ (500 mg, 0.15 mmol) was added to a 100 mL Parr reactor in a glove box. CO₂ (35 bar, 3.5 MPa) gas was charged and the mixture was stirred for hour at 325° C. and then and H₂ (1 bar, 0.1 MPa) gas was added to the same mixture, and the reaction was continued for an additional 2 hours. The reaction mixture was cooled to room temperature by applying cool air to the reactor. The reactor was depressurized and the reaction mixture analyzed. The reaction mixture contained cesium oxalate and cesium bicarbonate. Methanol (5 mL) was then added to the reactor, and the reactor was pressurized with CO₂ (35 bar, 3.5 MPa). The mixture was heated to 150° C., stirred overnight, and then depressurized. The remaining solvent (methanol) was removed by evaporation under vacuum. The product composition was analyzed and identified as being a mixture of dimethyl oxalate, cesium formate, and cesium bicarbonate. The overall yield of DMO was 58% and the yield of cesium formate as a byproduct was about 4% to 5%. ¹³C NMR (CD₃OD, in ppm): 53 (—OMe), 158 (—CO—), 161 (CsHCO₃), and 171 (CsHCOO).

1. A process for producing a disubstituted oxalate, the process comprising contacting a cesium salt with one or more alcohols and carbon dioxide (CO₂) under reaction conditions sufficient to produce a composition comprising a disubstituted oxalate having a general structure of: 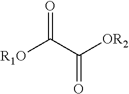
where R₁ and R₂ are each independently an alkyl group, a substituted alkyl group, an aromatic group, or a substituted aromatic group.

2. The process of claim 1, wherein the reaction conditions comprise a temperature of 125° C. to 200° C.

3. The process of claim 1, wherein the reactions conditions comprise a pressure of 2 MPa to 5 MPa.

4. The process of claim 1, wherein the cesium salt is cesium oxalate.

5. The process of claim 4, wherein the cesium oxalate is obtained by contacting 1) CO₂ and carbon monoxide (CO), under reaction conditions sufficient to form a composition comprising the cesium oxalate

6. The process of claim 5, wherein the reaction conditions for obtaining the cesium oxalate comprise a reaction temperature of 200° C. to 400° C.

7. The process of claim 5, wherein the reaction conditions for obtaining the cesium oxalate comprise providing carbon dioxide at a pressure of 2.0 MPa to 3.0 MPa, and providing carbon monoxide at a pressure of 1.0 MPa to 3 MPa.

8. The process of claim 7, wherein the carbon dioxide is contacted with the cesium carbonate at a reaction temperature of 200° C. to 400° C. for at least 1 hour to obtain a cesium carbonate/carbon dioxide reaction mixture, and contacting the cesium carbonate/carbon dioxide reaction mixture with the H₂ at the reaction temperature.

9. The process of claim 8, wherein formation of sodium formate is inhibited.

10. The process of claim 5, wherein the reaction conditions for obtaining the cesium oxalate comprise providing CO at a pressure of 2 MPa to 6 MPa, preferably about 2 MPa, and providing O₂ at a pressure of 0.05 MPa to 5 MPa.

11. The process of claim 5, further comprising isolating the cesium oxalate salt from the product stream prior to converting it to the disubstituted oxalate.

12. The process of claim 5, wherein the process is a single reactor synthesis such that cesium oxalate is generated in situ and then contacted with the one or more alcohols and additional CO₂ to produce the disubstituted oxalate.

13. The process of claim 1, wherein R₁ and R₂ each independently comprise 1 to 20 carbon atoms.

14. The process of claim 13, wherein R₁ and R₂ are each methyl groups.

15. The process of claim 1, further comprising isolating the disubstituted oxalate from the product stream.

16. The process of claim 15, further comprising reacting the disubstituted oxalate under conditions sufficient to form oxalic acid or ethylene glycol.

17. A composition for producing a disubstituted oxalate, the composition comprising a cesium salt, carbon dioxide, carbon monoxide, and an alcohol.

18. The composition of claim 17, wherein the alcohol is methanol and the disubstituted oxalate is dimethyl oxalate.

19. A disubstituted oxalate produced by the process of claim 1.

20. The disubstituted oxalate of claim 19, wherein the disubstituted oxalate is dimethyl oxalate.
